(19) United States
(12) Patent Application Publication
(10) Pub. No.: US 2008/0089870 A1
(43) Pub. Date: Apr. 17, 2008

(54) Cultured Melanocytes on Bioploymer Membranes for Treatment of Hyper and Hypopigmentation Disorders

(75) Inventors: Deepa Ghosh, Navi Mumbai (IN); Sudheer Shenoy, Navi Mumbai (IN); Pushpa Kuchroo, Navi Mumbai (IN); Viru Shah, Navi Mumbai (IN)

Correspondence Address: FOLEY AND LARDNER LLP;SUITE 500, 3000 K STREET NW, WASHINGTON, DC, 20007, US

(73) Assignee: Reliance Life Sciences PVT. LTD.

(21) Appl. No.: 11/872,431

(22) Filed: Oct. 15, 2007

(30) Foreign Application Priority Data
Oct. 13, 2006 (IN) 1697/MUM/2006

(51) Int. Cl. A61K 45/00; A61P 17/00; C12N 5/08

(52) U.S. Cl. 424/93.7; 435/371

(57) ABSTRACT

The present invention relates to a graft wherein cultured autologous melanocytes are delivered using a biopolymer. The present invention describes the composition, method of preparation and its properties relating to safety and efficacy. The graft of the present invention has a potential use in treating hyper- and hypopigmentation disorders.

### CROSS-REFERENCE TO RELATED PATENT APPLICATIONS

This application claims the benefit of Indian provisional application No. 1697/MUM/2006, filed on Oct. 13, 2006, the disclosure of which is incorporated by reference in its entirety.

### FIELD OF THE INVENTION

The present invention relates to a delivery system of skin melanocytes on a suitable carrier system, and its method of preparation. In one embodiment, the invention relates to grafts of autologous melanocytes, its methods of preparation, transportation to hospitals and their therapeutic applications including vitiligo.

### BACKGROUND OF THE INVENTION

Skin and Physiological Role of Melanocytes

The skin is the largest organ of the body and is composed of an external epithelial component called the epidermis separated from an underlying connective tissue component, the dermis, by a basement membrane. The portion of the dermis adjacent to the epidermis is called the reticular dermis and is composed primarily of collagen fibers produced by fibroblasts, microvessels and a few migrating leukocytes. The reticular dermis supplies all of the nutrition to the epidermis, which is devoid of blood vessels. The great majority of cells in the epidermis are keratinocytes, which are arranged in stratified layers. At the dermal-epidermal junction is the stratum basale, a single layer of keratinocytes with a small number of interspersed melanocytes (approximately 1 melanocyte per 30 keratinocytes) which supply melanin to nearby keratinocytes via dendrites projecting from the melanocyte. Skin color is determined by the absolute and relative amounts of different melanin types, of which the two major forms are eumelanin and pheomelanin. These are regulated by the melanocortin 1 receptor (MC1R). Variant MC1R sequences are associated with different skin color. The binding of melanocyte stimulating hormone (MSH) to MC1R results in the formation of eumelanin while the binding of the agouti protein to MC1R leads to the production of pheomelanin. Tanning of the skin due to UV exposure represents an increase in the content of eumelanin within the epidermis and its major purpose is increased photoprotection.

Hyperpigmentation Disorders

Disorders of hypo- and hyperpigmentation can result from a change in the number of melanocytes or a decrease or increase in the activity of the melanocytes. One of the most common disorders of hyperpigmentation is melasma. Exposure to the sun plays a very important role in the induction and maintenance of hyperpigmentation. Without protection, UV radiation can cause the cell's genetic coding or DNA to mutate. This can significantly alter cell functions, resulting in changes associated with rosacea, aging skin, compromised immune function and cancer.

In addition, inflammation resulting from rosacea, laser therapies, UV exposure, or other factors, can affect the functioning of the melanocytes, resulting in an abnormal increase in the production of melanin. This may be visually manifested as freckles, uneven patchy coloring or solar lentigos (often referred to as age spots). Medical research has focused on methods of regulating melanin in order to control abnormal production and resolve the appearance of hyperpigmented lesions. The present invention, in some embodiments, seeks to resolve hyperpigmentation by replacing overactive melanocyotes with transplanted, normally active melanocytes.

Hypopigmentation Disorders

Disorders associated with hypopigmentation include vitiligo, also known as leukoderma, which is often seen in association with inflammatory disorders of the skin e.g., atopic dermatitis.

Vitiligo (vit-ill-eye-go) is a pigmentation disorder in which melanocytes in the skin, the tissues that line the inside of the mouth and nose and genital and rectal areas (mucous membranes), and the retina of the eyes are destroyed. As a result, white patches of skin appear on different parts of the body. The hair that grows in areas affected by vitligo may turn white.

Vitiligo affects approximately 1 out of every 100 people in India and approximately 1 in 2000 in the USA. About 1 to 2 percent of the world's population, or 40 to 50 million people, have vitligo. Vitiligo can be found in all parts of the world. It affects all ethnic groups, but is far more disabling in those who have dark skin. This disease affects males and females equally. The usual age of onset is between 10 and 30 years old, but the condition can start at any age. Ninety-five percent of people who have vitiligo develop it before 40 years of age. The change in appearance caused by vitiligo can affect a person's emotional and psychological well-being and may create difficulty getting or keeping a job. People with this disorder can experience emotional stress, particularly if vitiligo develops on visible areas of the body such as the face, hands, arms, feet, or on the genitals. Adolescents, who are often particularly concerned about their appearance, can be devastated by widespread vitiligo. Some people who have vitiligo feel embarrassed, ashamed, depressed, or worried about how others will react.

The goal of treating vitligo is to restore the function of the skin and to improve the patient's appearance. Current therapy for vitiligo often takes a long time, for example, 6 to 18 months. The choice of therapy depends on the number of white patches and how widespread they are, and on the patient's preference for treatment.

Pathogenesis of Vitiligo

To date, three important hypotheses have been advanced to explain the pathogenesis of vitiligo. The autoimmune hypothesis originated from the observation that vitiligo is associated with some autoimmune diseases. Both cellular and humoral factors responsible for autoimmune damage to melanocytes have been demonstrated. The autocytotoxic or self-destruct hypothesis suggests that some toxic molecules produced during the biosynthesis of melanin are responsible for melanocyte damage in susceptible individuals. The neural hypothesis postulates that neurochemicals liberated from nerve endings are toxic to melanocytes. Some people have reported that sunburn or emotional distress can trigger vitiligo. Multiple mechanisms may be responsible for vitiligo.

Treatments Available

There is no universally effective medical or surgical treatment for vitiligo; although a number approaches are known to be effective. In addition to these medical and surgical therapies that are listed later in the text, one has to always keep in mind adjunct therapies, such as broad-spectrum sunscreens to prevent photo damage of vitiliginous skin, and cosmetic camouflage of disfiguring skin with stains or make-up in exposed areas of vitiligo.

While there are a number of topical agents that inhibit the synthesis of melanin, researchers are focusing on breakthroughs that directly control the output of the tyrosinase enzyme resulting in greater control over pigmentary changes. Topical skin care preparations that assist in increasing the rate of cellular turnover and normalization of the epidermis also can lessen the appearance of abnormally pigmented areas because newer cells appear to contain fewer pigment granules. However, preliminary investigations have reported repigmentation of vitiliginous lesions with such topical skin care preparations containing isoprinosine, levamisole, suplatast tosilate, cyclosporine, all of which are immunosuppressants/immunomodulators.

Medical Treatments

Topical steroid therapy are helpful in re-pigmenting (returning the color to white patches) the skin, particularly if started early in the disease. Patients need to apply the steroid topical cream to the white patches on their skin for at least 3 months before seeing any results. There is also a risk of side effects such as skin shrinkage and skin striae (streaks or lines on the skin) for which the doctor has to closely monitor.

Psoralen UVA photochemotherapy (PUVA) involves taking psoralen orally or topically, followed by carefully timed exposure to ultraviolet A (UVA) light from a special lamp or to sunlight. Patients usually receive treatments in their doctors' offices so that they can be carefully watched for any side effects. Patients must minimize exposure to sunlight at other times. However, it is time-consuming and care must be taken to avoid side effects, which can sometimes be severe. Psoralens are drugs that contain chemicals that react with ultraviolet light to cause darkening of the skin. There are two major potential side effects of topical PUVA therapy: (1) severe sunburn and blistering and (2) too much re-pigmentation or darkening of the treated patches or the normal skin surrounding the vitiligo (hyperpigmentation). Other known side effects of oral psoralen include nausea and vomiting, itching, abnormal hair growth, and an increased skin cancer risk.

De-pigmentation involves fading the rest of the skin on the body to match the already white areas, and is more often used on people with extensive vitiligo. Patients apply the drug monobenzylether of hydroquinone (monobenzone or Benoquin) twice a day to pigmented areas until they match the already depigmented areas. Patients must avoid direct skin-to-skin contact with other people for at least 2 hours after applying the drug. The major side effect of de-pigmentation therapy is inflammation (redness and swelling) of the skin. Patients may also experience itching, dry skin or abnormal darkening of the membrane that covers the white of the eye. De-pigmentation is permanent and cannot be reversed. In addition, a person who undergoes de-pigmentation will always be abnormally sensitive to sunlight.

Surgical Treatments

Several surgical procedures for the treatment of de-pigmented skin have been reported to be effective in patients who have experienced difficulty in receiving good responses from medical treatments. The surgical therapeutic approaches include skin grafting. During skin grafting, the doctor removes sections of the normal, pigmented skin (donor sites) and places them on the de-pigmented areas (recipient sites).

Surgical techniques include:

1. Suction Blister grafts are created on patient's normally pigmented skin using heat, suction, or freezing. The tops of the blisters are then cut out and transplanted to a de-pigmented skin area. The risks of blister grafting include the development of a cobblestone appearance, scarring, and lack of re-pigmentation. However, there is less risk of scarring with this procedure than with other types of grafting.

2. Punch grafts have been used to replace de-pigmented skin. Punch grafts are small pieces of normal skin that is removed from the patient using punches that are available commercially. Following dermabrading of the vitiliginous skin, these grafts are placed over the area to induce pigmentation. This technique, although effective, leads to a cobblestone appearance and scarring.

3. Tattooing works best for the lip area, particularly in people with dark skin; however, it is difficult for the doctor to match perfectly the color of the skin of the surrounding area. Tattooing tends to fade over time. In addition, tattooing of the lips may lead to episodes of blister outbreaks caused by the herpes simplex virus.

Other Methods

Additional methods for the treatment of vitiligo have been proposed in several patents and patent applications, including U.S. Pat. Nos. 4,866,038, 5,700,450, 6,039,972, 6,660,305, and 6,673,603, U.S. published patent application Nos. 2001/0006813 and 2002/0106353, and PCT publications WO2004096981A2 and WO2005011676A 1. Some of these documents propose compositions for stimulating melanin and/or melanocyte production. Another proposes direct injection of melanin into the skin.

Other documents are focused on means of delivering cells. For example, a cell paste comprising keratinocytes and fibroblasts; cultured melanocytes and keratinocytes that are delivered using a carrier matrix such as a fibrin glue; or delivery of a cell suspension with a specially adapted device. Another describes a wound dressing containing mammalian cells anchored on hydrophobic synthetic polymer film. With these wound dressings, the films are applied as such to the skin and the cells need to migrate through the perforations. This type of delivery leads to less cells being directly in contact with the skin and the efficiency of the porous nature of the films is critical for migration of the cells. Another document proposes a method of increasing pigmentation comprising the implantation of skin grafts on the vitiliginous patches, with the skin grafts comprising melanocytes activated by contact with an effective amount of a diacylglycerol. With this methodology, the donor skin area to be taken for grafting depends upon the de-pigmented area that needs to be covered by the skin graft, i.e., a larger area of donor skin would be required for the coverage of large de-pigmented areas. Other techniques include pure melanocyte cultures, and cocultures of melanocytes and keratinocytes. These techniques are beneficial for localized lesions.

Recent advances in culturing human melanocytes now make it possible to transplant autologous melanocytes obtained from areas of normal skin color into areas of skin that are de-pigmented. In direct melanocyte transplant procedures, a sample of the patient's normally pigmented skin is taken and subjected to enzymatic dissociation. The resulting cell suspension that includes keratinocytes as well as melanocytes is then directly added to de-pigmented areas that have been debrided.

The disadvantages of such methods include:

(a) Cell number identification: As the resulting cell suspension contains all types of epidermal skin cells, the identification of the number of melanocytes administered at the de-pigmented site remains unclear.

(b) Retaining cells at the de-pigmented site: As the cells are sprayed or poured on the de-pigmented area, cells can become lost in subsequently applied dressing. Hence, the number of cells retained on the debrided area is unclear.

(c) Area coverage: If the initial biopsy taken is small compared to the de-pigmented area, then the number of melanocytes administered to the de-pigmented site would not be sufficient to produce satisfactory levels of pigmentation.

Some of the above disadvantages are overcome by developing a method to culture melanocytes in vitro. Following the multiplication of melanocytes in the culture dish, the cells are dissociated from the dish and the cells transplanted onto the patient's debrided de-pigmented skin patches. To date, all studies on melanocytes transplantation have been conducted either in hospitals or in medical centers attached to research centers, and involve the delivery of a melanocyte suspension to the skin. This delivery method is inefficient, with cells not being correctly delivered to the wound bed or becoming trapped in the gauze applied over the treated skin.

Although strides have been made into understanding the pathogenesis, and treatment of vitiligo, many of the commercially available products are only marginally effective and rarely achieve an even-looking skin tone.

Looking to the need of the hour, the present invention provides a novel system to deliver functional melanocytes at the vitiliginous areas to induce repigmentation, in a manner that overcomes the disadvantages of existing cell delivery systems.

The present invention provides grafts of functional melanocytes for transplantation, which can be transported from a centralized manufacturing or processing center to a doctor or hospital located at a different location. The melanocytes as developed under the present invention are meant for delivery at vitiliginous areas of the skin to induce repigmentation. Previous studies have all been carried out in hospitals attached to research centers.

To date, it has not been possible to conduct transplantation of melanocytes remotely, due to the inability to safely and reliably transport the melanocyte cultures over distances. Thus, there remains a need to have a system that would enable cells to be processed in a centralized facility and delivered to various hospitals. Retaining the viability of the cells during transport is critical for the success of grafting. The inventors of the present invention have developed a system that focuses on the biopsy transport; melanocytes culture, its transport and delivery on a biopolymer. The delivery system of the present invention provides cells to de-pigmented skin in a manner that facilitates melanocyte migration and colonization in the skin, resulting in re-pigmentation.

The present invention succeeds in generating such a system, which will greatly reduce the cost and increase the ease of therapy, therefore benefiting patients.

It is proposed that using grafts of autologous cultured melanocytes (optionally in addition with keratinocytes), as developed by the present invention will achieve pigmentation with good healing thereby alleviating hypopigmented conditions such as vitiligo. The invention provides the advantage of a pre-packaged “ready-made” composition that can be shipped directly to a hospital or doctor from a manufacturing facility, rather than rely on the hospital, a nearby research center or doctor, to assemble or prepare the composition. This advantage greatly reduces costs, and increases efficiency and easy of use.

### SUMMARY OF THE INVENTION

The present disclosure relates to a graft comprising proliferative melanocytes cultured on a transparent biocompatible film. The cells are directly delivered to the de-pigmented site by inverting the film so that the cells are in opposition to the debrided site. In one embodiment, the graft of the present invention retains the viability of the melanocyte cells for a minimum of 96 hours under transport conditions before grafting. The present invention is useful for treatment of hypopigmentation disorders such as vitiligo/leucoderma, and also hyperpigmentation conditions such as Nevi.

In one embodiment, hyper pigmented area are debrided leading to the removal of hyperactive melanocytes which are responsible for very dark pigmentation. Transplantation with normal melanocytes at this site is then used to provide normopigmentation.

In one embodiment, the invention comprises a graft composition capable of inducing pigmentation in skin comprising melanocyte cells derived from autologous epidermis and a biopolymer membrane, wherein at least 75% of the cells remain viable in the composition for at least 72 hours under transport conditions at 5-37° C. In related embodiments, at least 80% of the cells remain viable in the composition for at least 96 hours under transport conditions at 5-37° C.

In additional embodiments, the melanocyte compositions further comprise keratinocytes. In some embodiments, the ratio of melanocyte to keratinocyte is from 0.8:2 to 1:1.

In the grafts of the invention, the cells may be in a monolayer. In some embodiments, the monolayer is at least 25%, at least 50%, at least 70%, at least 90%; and 100% confluent (i.e., ranging from semi-confluent to confluent).

In one embodiment, the melanocytes of the invention are capable of further proliferation. In a related embodiment, at least 90% of the melanocytes in the graft are actively proliferating melanocytes.

The cells of the invention are grown on a biologically compatible polymer (biopolymer) membrane. Suitable biologically compatible membranes include biodegradable, natural biocompatible and synthetic biocompatible membranes. In some embodiments, the biopolymer membrane is selected from the group consisting of polylactic acid (PLA), polyglycolic acid (PGA), and polylactic-polyglycolic acid copolymer (PLGA) having a molecular weight of at least 50,000 Da.

Therefore, in related embodiments, the invention comprises a composition comprising a sub-confluent monolayer of melanocyte cells (optionally co-cultured with keratinocytes) and a biopolymer membrane, wherein at least 75% of the melanocyte cells remain viable in the composition for at least 72 hours under transport conditions at 5-37° C.

The compositions of the invention are suitable for therapy of hypo-pigmentation disorders of the skin. Accordingly, in one aspect, the melanocyte cells are present in a sufficient quantity so that the resulting composition has a therapeutic effect against a hypo-pigmentation disorder of the skin.

The invention also includes a process for preparing compositions of melanocytes on biopolymer membranes suitable for treatment of hypopigmentation disorders. In one aspect, such a process comprises:
a) isolating melanocyte cells from autologous epidermis;
b) reparing a cell suspension comprising the isolated melanocyte cells;
c) expanding the melanocyte cells;
d) optionally, cryopreserving the melanocyte cells;
e) optionally, thawing the melanocyte cells;
f) seeding the melanocyte cells onto a biopolymer membrane;
g) expanding the cells into a sub-confluent monolayer on the biopolymer membrane; and
h) transporting the biopolymer membrane using a transport device comprising transport media;
wherein at least 75% of the cells remain viable in the composition for at least 72 hours under transport conditions.

In related embodiments, the biopolymer membrane comprises polylactic acid (PLA), and the melanocyte cells are seeded onto the biopolymer membrane at a cell concentration of 1×10⁴ to 1×10⁵ cells/cm². In further embodiments, the transport device comprises polycarbonate. In another, the transport media comprises carbon dioxide enriched media. In one embodiment, the transport medium is KSFM media.

The invention also provides a method for treating an individual requiring skin pigmentation. In one embodiment, the individual is human. In further embodiments, the individual is affected with a hypopigementation disorder, such as leucoderma or vitiligo. In related embodiments, the method comprises applying a graft composition capable of inducing pigmentation in skin comprising melanocyte cells derived from autologous epidermis and a biopolymer membrane, wherein at least 75% of the cells remain viable in the composition for at least 72 hours under transport conditions at 5-37° C. In related embodiments, at least 80% of the cells remain viable in the composition for at least 96 hours under transport conditions at 5-37° C.; wherein the composition enhances the rate of epidermal regeneration in the skin of the individual.

Accordingly, the invention provides a method for treating vitiligo, in a subject, comprising using a biopolymer membrane as a delivery system, and delivering to the skin a sub-confluent monolayer of melanocyte.

In other embodiments, the invention provides a kit for treating vitiligo, wherein the kit comprises (1) a composition comprising (a) a sub-confluent monolayer of melanocyte cells and (b) a biopolymer membrane, wherein the monolayer is on the biopolymer membrane; and (2) a transport device, wherein at least 75% of the melanocyte cells remain viable in the composition for at least 72 hours under transport conditions at 5-37° C.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1: Illustrates the interactive biopolymer graft of the present invention comprising cultured melanocytes on PLA sheets.

FIG. 2: Illustrates the schematic representation of the transport container for the wound cover of the present invention. The design of the transport container is registered under the Indian design patent act having number 198591 which is incorporated herein by reference.

FIG. 3: Illustrates the comparative result of the mycoplasma testing of the cells as shown in 3A: Mycoplasma positive control; 3 B: Cultured melanocytes showing absence of mycoplasma.

FIG. 4: Illustrates the cell identity testing of the melanocytes by MEL-5 antibody staining wherein, the melanocytes are identified as green cells. The cell nuclei are stained with DAPI.

FIG. 5: Illustrates the MART-1 gene expression in melanocytes by PCR analyses.

FIG. 6: Illustrates the functionality of cultured melanocytes by DOPA staining.

FIG. 7: Illustrates the migration of melanocytes from PLA sheets to the wound cover indicated by the presence of red fluorescent-labeled cells in the wound bed.

FIG. 8: Table:1. Illustrates the results of the in vitro tumorigenesis assay indicating the safety of the cultured melanocytes for transplantation.

FIG. 9: Illustrates the viability of melanocytes under transport conditions.

FIG. 10: Co-culture of melanocytes plus keratinocytes under phase contrast microscope.

FIG. 11: Identification of keratinocytes by keratin staining.

FIG. 12: Identification of MHC-II (HLA-DR) expression in keratinocytes by-PCR in case allogeneic keratinocytes are used.

FIG. 13: Illustrates the results of the in vitro tumorigenesis assay indicating the safety of the cultured keratinocytes for transplantation.

FIG. 14: Illustrates the temperature change over time during transport in specially adapted transport containers.

### DETAILED DESCRIPTION OF EMBODIMENTS OF THE INVENTION

The present invention provides a composition and a delivery system comprising a cultured graft of actively growing epidermal cells such as melanocytes, or a melanocyte-keratinocytes mixture, on a biocompatible support material such as PLA, and suitable for delivery from a centralized processing center to hospitals located in various other location where they are transplanted.

In one embodiment, the process of preparing the graft involves the optimization of scaffolds onto which cells are seeded. Scaffolds can be selected from the group comprising of natural materials such as collagen and fibrin or synthetic materials such as degradable polyesters used in surgical sutures. Scaffolds take forms ranging from sponge-like sheets and fabrics to gels to highly complex structures with intricate pores and channels made with new materials processing technologies. The spatial and compositional properties of the scaffold, the porosity of the scaffold and interconnectivity of the pores are all required to enable cell penetration into the scaffold as well as the transport of nutrients and waste products.

In another embodiment, the melanocytes that form part of the differentiated keratinocytes cell culture process are delivered as 4-5 layer thick tissue, which can then be enzymatically detached from the culture dish and transplanted onto the de-pigmented area following debridement.

In certain embodiments, the melanocytes can be cultured directly onto a delivery membrane in a culture vessel, which is then peeled off when required for use.

In another embodiment of the present invention, the graft of cultured melanocyte on biopolymer include genetic modification of transplanted cells to improve pigmentation.

Biocompatible polymers can be either natural or synthetic. In general, synthetic polymers offer greater advantages than natural materials in that they can be tailored to give a wider range of properties and more predictable lot-to-lot uniformity than can materials from natural sources. Synthetic polymers also represent a more reliable source of raw materials, one free from concerns of immunogenicity and infectivity.

Polylactic acid (PLA), polyglycolic acid (PGA), polylactic-co-glycolic acid (PLGA), and polyurethane are some of the preferred biocompatible polymers in this invention. These polymers have the advantage of not requiring surgical removal after they serve their intended purposes. These polymers have found a broad range of pharmaceutical and biomedical applications based on their unique properties, including versatile degradation kinetics, non-toxicity, and biocompatibility. PGA is a highly crystalline polymer and the most hydrophilic among them. It has a very high melting point (224° C. to 226° C.), and the degradation rate of PGA is much higher than that of PLA. Random PLGA copolymers with different ratios of lactide (LA) and glycolide (GA) exhibit different degradation rates, and thus can be tailor-made for specific applications requiring specific degradation kinetics ranging from weeks to months. They are generally more amorphous than their homo-polymers and become most susceptible to hydrolysis when the two-monomer contents are the same. Polylactic acid (PLA) and polyglycolic acid (PGA) are among the few synthetic degradable polymers that have been approved for clinical use and these have been widely studied in tissue development.

Segmented polyurethane elastomers have a wide use as biomaterials due to their excellent mechanical properties and great chemical versatility. Research devoted to the development of biomedical polyurethanes has primarily focused on long-term applications such as vascular grafts and pacemaker lead insulators. The polyurethane polymer is used in a number of different forms and in a range of applications, both in the biomedical field and others. The material is fabricated by casting or other molding techniques to form a substrate, which can be used along or combined with other substrates to form homogenous multi-layered materials. Such multilayered homogeneous polyurethane materials may be formed with layers having different degrees of degradability.

The factors affecting the mechanical performance of biodegradable polymers include monomer selection, initiator selection, process conditions, and the presence of additives. These factors, in turn, influence the polymer's hydrophilicity, crystallinity, melt and glass-transition temperatures, molecular weight, molecular-weight distribution, end groups, sequence distribution (random versus blocky), and presence of residual monomer or additives. In addition, the polymer scientist working with biodegradable materials must evaluate each of these variables for its effect on biodegradation. Although the characteristics of these polymers are well understood, they merely serve to provide a 3-D biocompatible structure onto which cells can attach and do not interact with the cells. In the body, cells are situated within an extracellular matrix (ECM), which provides tissues with the appropriate architecture as well as signaling pathways that influence key cell function such as migration, proliferation, and differentiation. Research continues on the utilization of matrix molecules along with specific growth factors to optimize cell adherence to the scaffolds and direct cell activity.

Culturing melanocytes on the present delivery system avoids the disadvantages faced by existing delivery system for melanocytes.

The use of polymers under the present invention, on which the skin cells are cultured and transferred, improves the ease of graft handling during cell culture and its transplantation. The polymer used in the present invention being transparent permits microscopic observation of cells during cell culture as well as the visualization of the underlying skin after its application on the de-pigmented skin.

In one embodiment, the present disclosure provides the culture and transfer of human epidermal melanocytes for the treatment of de-pigmented skin. The present invention aims at using a biocompatible polymer in lieu of its reported properties. Biodegradable polymers contemplated for use in the present invention, for wound healing or wound care compositions include polyesters, poly (amino acids), polyester amides, polyurethanes, or copolymers thereof. Besides, the polymer sheets selected from PLA, PGLA, and polyurethane have the following advantages of ease of handling during culture and application on the wound bed, and its ability to stick to the contours of the wound. The sheets of PLA, PGA, PGLA and polyurethane are transparent, permitting visualization of the cells during culture as well as the pigmentation process after its application on the wound site. Further it possesses barrier properties, preventing microbial contamination of the wounds.

The present invention provides the use of sheets made of biocompatible, biodegradable polymer such as PLA as a delivery system for melanocytes as well as the ability of using this system as a tool to deliver proliferating melanocytes to the depigmented skin.

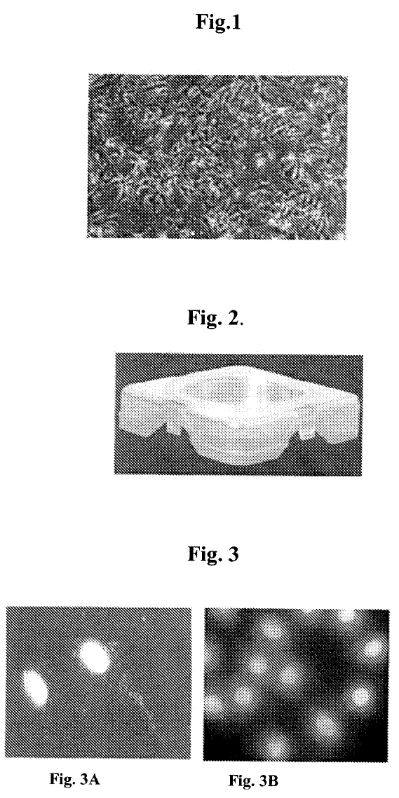

In one embodiment, the wound cover of the present invention primarily comprises cultured melanocytes on PLA sheets (FIG. 1) in a specially designed container (FIG. 2). The crude PLA is prepared by catalytic reaction using dilactide and is purified by re-dissolving in a solvent like acetone, chloroform which is then re-precipitated with water. In this process the unconverted dilactide, other monomers, and impurities will be removed along with some portion of catalyst used. The polymer films are made by using purified PLA.

The polymer film provides sufficient strength for the graft during culture process as well as its application. Unlike collagen sheets and fibrin sheets that are used for cell transplantation, the PLA films are not fragile and have sufficient strength. This property is beneficial during the manufacturing process i.e at the time of cell culture as well as handling of the graft during transplantation.

The present disclosure expects to make available biocompatible and biodegradable polymers to device designers and physicians that will help speed patient recovery. The present invention provides a composition of melanocytes.

In another embodiment, the present invention provides for the culturing of melanocytes along with other cells like keratinocytes and its delivery on a biocompatible polymer.

The present invention has provided a process for co-culturing and its use in transplantation in skin conditions such as vitiligo. The advantage of co-delivering keratinocytes with melanocytes is that it will enhance the rate of epithelialization of the affected skin and, by secreting growth factors, will accelerate pigmentation.

Most of the raw materials used in the preparation of the grafts of cultured melanocytes on biopolymer of the present invention are sterile products. The source of fetal bovine serum used for patient and/or donor skin transport is certified from BSE-free countries. All plasticware used in the manufacturing process are disposable and obtained from NUNC™ (USA) and Falcon™ (USA). In the grafts of cultured melanocyte on biopolymer of the present invention, PLA membrane serves as a carrier of melanocytes. PLA is cast into films and sterilized with ethylene oxide (ETO). The grafts of cultured melanocytes on biopolymer of the present invention are transported in specially designed polycarbonate dishes. As part of the transport container for the wound cover of the present invention, a silicone O-ring is used. All raw materials are tested to further ensure sterility of the materials.

The process of manufacturing the wound cover of the present invention is carried out in clean rooms under cGMP norms.

The following are involved in the preparation of the composition of graft of cultured melanocyte, under the present invention:

1. Isolation of melanocytes

2. Culture of melanocytes

3. Preparation of biopolymer films

4. Safety and efficacy studies.

The melanocytes cultured on the delivery system can then be transferred to the patient thus minimizing the handling of cells by the clinician. Since the cells are delivered on a film, the cells are subjected to minimal handling by the clinician. The graft of cultured melanocyte on biopolymer of the present invention is for use in conditions where patients have de-pigmented or hypopigmented skin such as vitiligo, leucoderma etc.

In one embodiment, the present invention uses PLA as the substrate to deliver melanocytes in view of its biochemical properties such as biocompatibility, controlled degradation rate, proven non-toxicity, high strength and controlled degradation rate. PLA films are semi-permeable, which makes them occlusive to liquid and bacteria, yet permeable to water vapor, oxygen, and carbon dioxide. Permeability is important, as the inventors have discovered that CO₂ rich media, such as KSFM, unexpectedly increased viability of melancytes on this substrate. PLA's adherance to the wound bed facilitates the migration of the cells to the wound bed. The PLA films being transparent helps in microscopic evaluation of cells during cell culture and visualization of the wound bed after grafting. Also the polymer films also ease graft handling during its application as well as permit grafts to be trimmed to fit vitiliginous lesions.

The maintenance of the viability of the biopsy is reflected in the viability of the cells obtained during its processing. The greater the viability of the isolated cells, better are the chances of melanocytes survival and proliferation. The present invention ensures the viability of the biopsy during shipping of the biopsy from the hospitals to the central processing center. Following excision, the skin biopsies in transport media retain about 80% of their viability for 96 hours when stored at 5-37° C. The insulated boxes filled with cool packs, maintain 5-25° C. under the transport conditions for 96 hours. Thus the viable cells could be isolated from biopsies that were in transit for 72 hours. The viability of the graft can be retained for about 96 hours under transport conditions, thereby facilitating its delivery to various hospitals located far away from the central processing facility.

Similarly, the present invention has provided transport conditions for maintaining the viability of cells from the culture-processing center to the hospitals for transplantation. The present invention has provided ideal storage media, shipping conditions and the duration of transport to ensure viability of the cells during the transit. During shipping the cultures are sealed to prevent contamination and media leakage. The present invention provides optimized conditions for transit wherein, for example, 80% of the cells retain their viability in presence of CO₂ enriched media for 96 hours when shipped at 8-25° C.

One example of a CO₂ enriched media is KSFM. (Available from Gibco-BRL specialty media. See Daley, J. P., Epstein, D. A., and Hawley-Nelson, P. (1990) Focus® 12, 68; Donovan, J. (1998) Focus 20, 38). KSFM media includes, for example, keratinocyte-SFM (basal medium) (500 ml) containing L-glutamine; bovine pituitary extract (25 mg); and recombinant epidermal growth factor (rEGF) (2.5 μg).

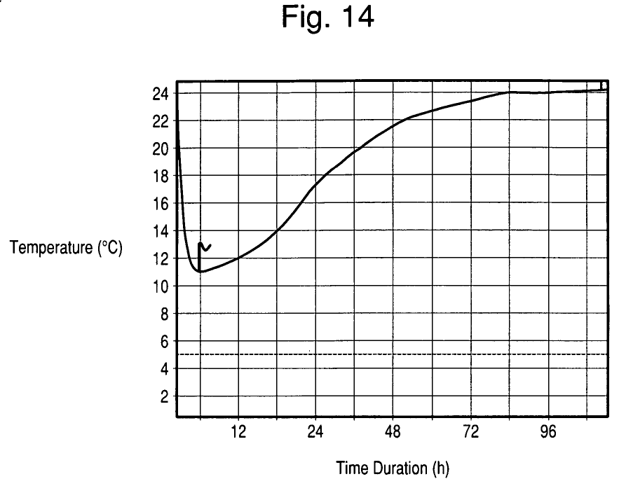

Melanocytes are cultured in 254-CF medium, a specialized melanocyte medium which support their proliferation. The cells are cultured in incubators that maintain temperature of 37° C. During shipping, however, the temperature is maintained between 8-25° C. for a period of 96 h. See e.g. FIG. 14. Melanocytes in melanocytes growth media are unable to retain their viability when exposed to the shipping temperatures. However, when the cells are cultured in KSFM media under regular conditions (37° C.) in the incubator, the cells retain their viability but the proliferation is not the same as in growth media. One aim is to maintain the viability as well as its function during shipping. It has been observed that melanocytes cultured in KSFM without CO₂ enrichment were not viable even for 24 h. However, melanocytes transported in CO₂ enriched KSFM remain viable for 96 h.

The present invention has provided a graft of cultured melanocyte on biopolymer as approximately 16 sq. cm. circular film. However, the size does not limit the scope of the invention, in that the graft can be of any size or shape. Further the present invention has provided grafts of 64 cm² from 1 cm² skin biopsy using the culture conditions with a seeding density of 3×10⁴/cm² in 35-40 days. Thus the present invention is able to propagate the melanocytes obtained from a small biopsy to cover large vitiliginous lesions.

For clinical use, the graft would be inverted on dermabraded skin. Dermabrasion is a method wherein the epidermis is removed from the skin using a specially adapted tool, known as a dermabrader. On inversion of the sheets on the dermabraded skin, the melanocytes migrate and form part of the regenerated epidermis. The presence of functional melanocytes in the regenerated epithelia results in pigmentation of the skin following exposure to sunlight or PUVA. The keratinocytes when presented as a cocultured graft also migrates onto the wound bed, colonizes and reconstitutes the epidermis. The growth factors secreted by the keratinocytes aid in inducing the recipient keratinocytes to migrate, proliferate and reform the epidermis resulting in early wound closure.

The presence of functional melanocytes in the regenerated epithelia will result in pigmentation of the skin following exposure to sunlight or PUVA. The keratinocytes when presented as a cocultured graft, will also migrate onto the wound bed, colonize and reconstitute the epidermis. The growth factors secreted by these cells aid in inducing the recipient cells to migrate, proliferate and reform the epidermis resulting in wound regeneration. Cultured melanocytes on PLA films could be delivered per se or as co-cultures along with autologous or allogeneic keratinocytes.

The following are exemplary embodiments of the invention. It should be appreciated by those skilled in the art that the techniques disclosed in the examples represent techniques discovered by the inventor to function well in the practice of the invention. However, those skilled in the art should, in light of the present disclosure, appreciate that changes can be made in the disclosed embodiments, and still obtain a like or similar result without departing from the spirit and scope of the invention.

### EXAMPLE 1

### Preparation of Cultured Melanocyte Graft on Biopolymer

Collection of the Skin

A punch biopsy was collected from the person who needs to be grafted hereto referred to as patient. Patient's blood (5 ml) was collected for infectious disease testing to determine the ID status of the patient to ensure safety to the operator as well as the patient at the time of transplantation. Skin biopsy collection vials containing DMEM, Iscove medium (Invirtrogen USA), 10% fetal bovine serum (FBS) (Hyclone, USA), antibiotic-antimycotic solution (Sigma USA) and gentamycin (Invitrogen, USA) were transported to hospitals in insulated boxes containing ice-packs. The insulated boxes were made of EPS (expanded polystyrene) and maintained at a temperature of 8-25° C. for 72 hours. The vials were stored in the refrigerator for a maximum of one week till use. The biopsies were collected in these transport vials and shipped back in insulated boxes for further processing within 48 hours of sample collection.

Preparation of the Polymer Films

The polymer film was prepared by solvent casting method. The film was allowed to dry by evaporation of solvent at ambient temperature and sterilized by ETO. A typical preparation of the biopolymer sheet for example is described in detail in Indian application number IN 205/MUM/2006, filed on Feb. 14, 2006. More precisely the present invention has used PLA that is prepared by co-polymerization of DL-Lactide and L-Lactide (wt/wt:50/50), having an intrinsic viscosity of 0.85. About 15-25% PLA solution in acetone was cast on stainless steel plates by spin coating and left overnight at 37° C. for drying. For cell culture, the films were cut into circles of 4.5 cm diameter and sterilized by ethylene oxide (ETO). The physical properties of ETO sterilized films were characterized using standardized methods (IS/ASTM) The barrier properties of the film against microbes such as bacteria, yeast and fungi were assessed by the ability of the films to prevent contamination of the underlying nutrient matrix.

Cell Culture Procedure

Melanocytes isolation and expansion involved the following steps:

1. The patient skin was trimmed of excess fat, and decontaminated by washing serially in 70% alcohol, povidone-iodine (Mundipharma, Switzerland) and antibiotic-antimycotic solution.

2. Depending upon the thickness of the dermis, the biopsies were incubated in 0.2-0.4% dispase (Sigma, USA) for 1-18 hours. After dispase digestion, the epidermis was separated from the underlying dermis and the melanocytes were released from the epidermis by enzymatic digestion with 0.05% trypsin-EDTA (Invitrogen, USA).

3. Following trypsinization, the trypsin activity was neutralized with soybean trypsin inhibitor and the released cells were resuspended in the commercially available melanocyte growth media, 254-CF (available from Cascade Biologics USA), which selectively induces melanocyte proliferation, but does not support keratinocyte proliferation. Because the epidermis is physically separated from the dermis after an enzymatic digestion, there is also no fibroblast contamination of the cell culture.

Cells were seeded in tissue flasks at density of 4×10⁴ cells/cm². The media was replaced every alternate day and the cells were passaged on reaching 70-80% confluency, and cell viability was assessed.

4. When the cells reached confluency, the cells were passaged. Depending upon the area of the skin to be covered, the cells were passaged 2-3 times to obtain sufficient cell numbers. Before dispatching the graft, the cells were trypsinized and seeded on biopolymer films (1×10⁴-1×10⁵ cells/cm² graft) in specially designed containers (Design Number 198591)

5. The cells were allowed to attach and proliferate.

FIG. 1 represents the biopolymer graft of the present invention wherein the cultured melanocytes are seen distributed on a PLA film.

In-Process Controls:

The safety of the cells to the recipient is ensured by continuously monitoring the sterility and endotoxin levels of the cells during the culture period.

Besides, to overrule that culture conditions produce abnormality in the cells, several batches of melanocytes were tested for in-vitro tumorigenicity as well as karyotyping. All experiments showed no abnormality in cultured cells.

Melanocyte cell growth on PLA films was analysed on days 2,3 and 4 after seeding by trypan blue dye exclusion method following trypsinization. Each experiment was done in triplicate.

In one experiment, normal melanocytes were isolated and cultured from punch biopsies (8 mm) that were obtained from 22 donors, age 25-63 yrs, following informed consent. On an average 1.6×10⁶/cm² viable cells (by trypan blue exclusion method) were isolated from the epidermis of each biopsy. Using earlier mentioned culture conditions, the inventors were able to culture melanocytes for greater than 3 passages in all patients irrespective of age, sex or presence or absence of vitiligo. In some cases it was able to culture cells till passage seven without a change in cell morphology. Longer duration to reach confluency was observed in donors of 45 years and above.

Human Transplantation

At least one hour prior to use, pouches containing melanocytes on a PLA backing are removed from the specially adapted transport container and allowed to stabilize at room temperature. The melanocytes on a PLA backing and the specially adapted transport container are described elsewhere herein. At the time of use, the sealed trays are opened under sterile conditions and the lid of the dish is carefully lifted, and the transport media is discarded through the notch indicated by an arrow on the dish. The graft containing the cells is gently lifted from the dish and rinsed with normal saline. The graft with the cells facing upwards is placed on the palm wearing sterile gloves. The graft is cut to the size of the lesions using a pair of forceps. The cut grafts are lifted from the edge and placed facedown on the dermabraded skin so that the cells are in apposition to the skin. Care should be taken not to drag the sheet over the wound bed. The graft is then finally covered with secondary dressings. The affected area should remain immobilized for at least 2-3 days and the wound area should not be subjected to any mechanical or frictional forces. The patients will be followed-up after 3, 6 and 9 months from the day of application of study device. Patients may be followed for longer period for the collection of safety and efficacy data.

Relevant features that are assessed includes repigmentation of the skin, both by a trained clinician and by photographic evaluation. The transplanted area is also assessed for hyperpigmentation, infection or breakdown of the transplant, and for any affect on patient vital signs.

### EXAMPLE 2

### Co-Culture Techniques

The application of keratinocytes to skin resulted in enhanced rate of epithelialization and improved the rate of healing of the wound that is produced following debridement of the de-pigmented skin.

Keratinocytes were obtained either from the patient undergoing melanocyte treatment or from another donor. FIG. 10 shows the co-cultured cells on a biopolymer. When using cultured keratinocytes obtained from the patient directly, keratinocytes will continue to survive for a longer time and form part of the reconstituted epithelia. The application of allogeneic cultured keratinocytes will enhance the rate of healing by stimulating the body to regenerate even if these cells only survive temporarily, however.

### EXAMPLE 3

### Transport of the Graft of Cultured Melanocyte Biopolymer

There are no previous reports of transportation of melanocytes beyond a hospital. The present invention demonstrates how such portability may be achieved.

An example design of a transport container, its assembly, and use in transport of cultured cells is presented in Indian patent application 60/MUM/2006, which is incorporated herein by reference. The design of the container is shown in FIG. 2. Sterilized PLA films were placed in these transport dishes and soaked in PBS for 1 hour. The film in each dish was held in place with polycarbonate ring. The cells were seeded with 3-5×10⁴ cells/cm² and cultured for 2 days before transport. Before dispatching to hospitals, the media in the dishes were replaced with CO₂ enriched culture media using a flow meter. Sterilized PLA films were placed in specially designed transport dishes (Ampson, India) and soaked in PBS for 1 h. The film in each dish was held in place with polycarbonate ring. The cells were seeded at 3-5×10⁴ cells/cm² and cultured for 2 days before transport. For shipping, the media in the transport dishes was replaced with CO₂ enriched media (1.0-1.5 μl/min for 100 ml) using a flow meter. The clasps on the dishes were closed securely to ensure viability and sterility of the grafts during transport. The dishes were then placed in insulated boxes that maintain a temperature between 8-25° C. for 96 h and shipped to various hospitals for transplantation. Insulated boxes used for transport simulation studies were validated using Temprecord data logger. (Temprecord International Ltd. USA). This study was done to determine the temperature the EPS BOXES maintained for a period of 96 h. The data logger was used to track the temperature inside the box for period of 96 h. See, e.g., FIG. 14.

Product Characterisation and Testing

### EXAMPLE 4

### Safety Testing

To ensure recipient and handler safety, the cells from donors before transport simulations were subjected to the following tests:

1. Karyotyping: Karyological analysis was conducted on cells to determine the number of chromosomes and check for the presence of abnormalities. There was no evidence of clinically significant numerical or structural chromosomal abnormalities.

2. Mycoplasma testing: This test was done by Hoechst staining. Using mycoplasma stain kit, the cells were examined under fluorescence staining wherein the positive cultures are identified by particulate or filamentous fluorescence around the cell nuclei (FIG. 3A) and the negative cultures are identified by only nuclear staining as indicated herein (FIG. 3B)

3. Testing for infectious diseases: To ensure patient's safety, the blood of the patient (collected at the time of biopsy collection) was subjected to infectious disease testing by ELISA method to check for HIV, HBV, CMV, HBSAg and Syphilis. The cells before dispatch to the hospitals/recipients were also tested for infectious disease by PCR (polymerase chain reaction) method.

4. Sterility: The sterility of the product was ensured by stringent in-process testing like bioburden, endotoxin testing by LAL method and sterility testing. The sterility testing includes the detection of aerobic and anaerobic microbes. The test was performed by inoculating test samples in two different sterile nutritive media namely Fluid Thioglycolate medium (FTM) and Soybean Casein Digest medium (SCDM). The results showed no growth in the inoculated media during the incubation period of 14 days which indicates the sterility of the sample. The presence of bacterial endotoxins was determined by the gel-clot technique using the Limulus Amoebocyte Lysate (LAL) reagent. When incubated at 37° C. for one hour in the presence of bacterial endotoxins the LAL reagent forms a firm gel clot. The bioburden test was conducted to determine the microbial load in the product which was determined in temr sof number of colonies appearing on plates of solide media. The media used in the test was Soybean Casein Digest Agar (SCDA).

### EXAMPLE 5

### Cell Characterization

1. Identification

The cultured cells were analyzed and identified by immunohistochemical analysis.

Melanocyte Graft

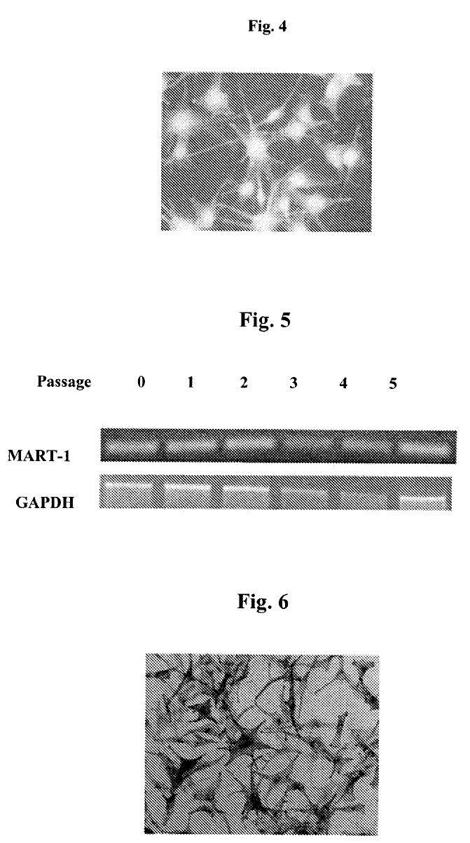

To determine the purity of melanocyte cultures, the cells were identified using antibodies to MEL-5. All cells test positive to MEL-5 antibody (FIG. 4). MEL-5 (Ta99) monoclonal antibody detects a differentiation-related, pigmentation-associated glycoprotein (gp75), (75 kD) expressed by melanoma cells, normal melanocytes and nevi. The gp75 antigen is now considered as the tyrosinase-related protein (TRP-1).

For the Co-Cultured Graft

To determine the identity and the number of keratinocytes in the graft, the cells were determined by immuno-staining the cells with PAN-keratin antibody that specifically binds to keratinocytes. The positive cells were detected using FITC-conjugated anti-mouse secondary antibody and DAPI was used to identify the nucleus of the cells as seen in FIG. 11.

Procedure: Cultured melanocytes were seed in two well chamber slides at 0.2×10⁴ cells/well. The cells were fixed with 4% paraformaldehyde (Sigma USA) for 20 minutes and washed twice with phosphate buffered saline (PBS). Following permeabilization with 0.2% Triton X-100 (Sigma, USA) for 1 hour, cells were stained with primary monoclonal Mel-5-antibody (Sigma, USA) for 1 hour, washed with PBS, and positive cells were detected with Alexa Fluor 488-conjugated goat anti-mouse (Molecular Probes, USA) secondary antibody. Cells were washed with PBS and incubated with DAPI (Sigma, USA) for 10 minutes at room temperature. Melanocytes were identified by the green fluorescence, while all cells in the field were detected by blue nuclei. The specificity of staining was determined with secondary antibody, alone which served as controls.

2. MART-1 PCR: As shown in FIG. 5, the expression of MART-1 in all passages of cultured melanocytes indicated the ability of the cultured cells to induce pigmentation due to production of the MART-1 protein. GAPDH (Glyceraldehyde phosphate 3-dehydrogenase) served as an internal control. MART-1 forms a complex with Pmel17 (a melanosomal protein) and affects its expression, stability, trafficking, and the processing which is critical to the formation of Stage II melanosomes. MART-1 is thus indispensable for Pmel17 function and plays an important role in regulating mammalian pigmentation. The RT-PCR technique involved the following steps: Total RNA from melanocytes at different passages was extracted with Trizol reagent (Invitrogen, USA) as per the manufacturer's protocol. Reverse transcription was performed with Superscript™. First-strand synthesis system for RT-PCR (Invitrogen, USA) as per the manufacturer's protocol. Gene amplification was carried out in 25 μL reaction volume each containing 1.5 μL of c-DNA in PCR Supermix (Invitrogen, USA). PCR conditions used were: 5 min at 95° C. followed by 95° C. for 45 sec, 55° C. for 45 sec and 72° C. for 45 sec with a final extension of 10 min at 72° C. Amplification was performed in a thermal cycler (Biometra, Germany) with the following specific primers designed from sequences obtained from Genbank.

MART-1 (240 bp):

(SEQ ID NO: 1)

5′- GCTCATCGGCTGTTGGTATT -3′ (sense),

(SEQ ID NO: 2)

5′- ATAAGCAGGTGGAGCATTGG -3′ (anti-sense);

Glyceraldehyde-3-phosphate dehydrogenase (GAPDH) (750 bp):

(SEQ ID NO: 3)

5′-GGG-CTG-CTT-TTA-ACT-CTG-GT-3′ (sense),

(SEQ ID NO: 4)

5′-GGG-CTG-CTT-TTA-ACT-CTG-GT-3′ (anti-sense).

The PCR products were separated on a 2% agarose gel and visualized under UV light (Image master, Amersham Biosciences, USA).

Levels of MART-1 expression remained unchanged in all passages of cultured melanocytes indicating the ability of the cultured cells at all tested passages to induce pigmentation. The present invention also tested the expression levels until passage five. GAPDH (Glyceraldehyde phosphate 3-dehydrogenase) served as an internal control for equal RNA loading.

3. Functional assay: The ability of cultured melanocytes to convert DOPA to melanin indicates the functional characteristics of the cells. Functional melanocytes produced melanin in a chemical reaction in which tyrosinase catalyses the conversion of tyrosine to DOPA which is further converted to melanin. The functional assay was conducted as follows: Cultured melanocytes before seeding on PLA films were cultured in chamber slides and fixed with 10% formalin in PBS for 3 hours at 4° C. Cells were rinsed with PBS and incubated with L-DOPA (0.05 mg/ml) in PBS for 3 hours at 37° C. Following incubation, the cells were rinsed with PBS and fixed with 10% buffered formalin for 1 hour. The functional melanocytes stained brown in the presence of L-DOPA. (FIG. 6).

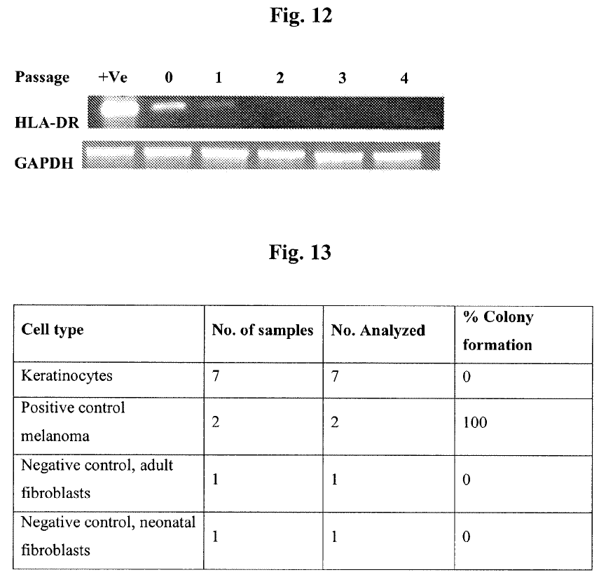

4. MHC-II (HLA-DR) expression: MHC-II plays an important role in the immune response. Downregulation of MHC markers is often associated with cancer. The identification of the MHC-class II (HLA-DR) expression in keratinocytes by PCR in cocultured product is illustrated in FIG. 12.

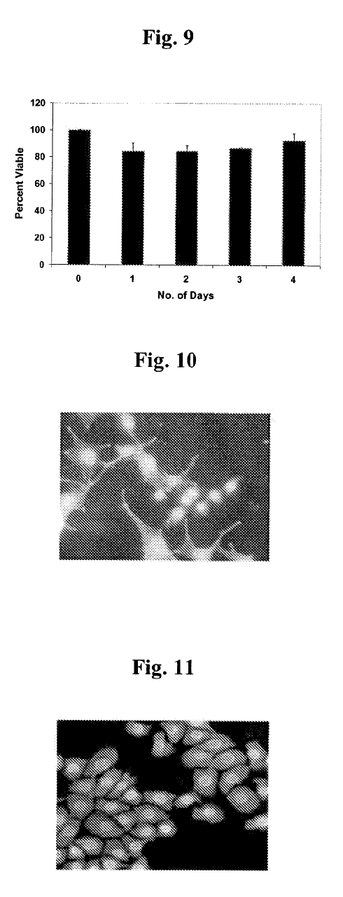

5. Viability of the cells under transport conditions: The viability of the melanocytes under transport conditions was assessed indirectly by the MTT method. Briefly, 0.5 mg/mL of 3-(4,5-Dimethylthiazol-2-yl)-2,5-diphenyltetrazolium bromide, also known as MTT (Hansen M B, Nielsen S E, Berg K., Re-examination and further development of a precise and rapid dye method for measuring cell growth and cell kill, J. Immol. Methods 1989 119:203-210), was added to the cells in triplicate dishes on days 0, 1, 2, 3 and 4 after shipment. Viable cells were indirectly determined by their ability to convert soluble MTT to insoluble formazan crystals. The crystals were solubilized and the absorbance was determined as the difference in optical density measured at a test wavelength of 570 nm and a reference wavelength of 650 nm (Shimadzu UV-VIS Spectrophotometer, Japan). With the assumption that all cells at the time of shipment (i.e., on day 0) were viable, the absorbance value at the time of shipment was assumed to be 100%. Viability is calculated at each time point as a percentage of absorbance compared with the absorbance at day 0. Under transport conditions, about 80% of cells retained their viability for about 96 h. It is assumed that within 96 h the cells under shipping conditions could be transported to tertiary hospitals without much loss in viability. A slight increase in viability observed at 96 h could be attributed to the temperature in the insulated box which had reached about 25° C. by 72 h. Proliferation of few cells might have started at this point, leading to increased cell numbers. The temperatures maintained by the insulated box during transport simulation condition were 11-24° C. for a period of 96 h. (FIG. 14). Cells on the graft maintained viability up to 4 days at a temperature of 8-35° C. (FIG. 9).

### EXAMPLE 6

### Efficacy Testing by Pre-Clinical Studies

1. Toxicity Testing:

PLA did not show any toxicity when tested on mice and guinea pigs. The entire graft also did not show any toxic effect in animals. The toxicity testing was done with a Dulbecco's Phosphate buffered saline) PBS solution with the PLA sheet for 72 h at 37° C. (hereinafter “test substance”). All toxicity studies were conducted in accordance with the Good Laboratory Practices (GLP) principles as published by OECD in 1998.

Acute intracutaneous toxicity studies were conducted using the contact solution in male and female New Zealand white rabbits. The test was performed by intracutaneous administration of 0.2 ml test substance (contact solution) at five sites on one side of each rabbit. Similarly 0.2 ml of distilled water (control) at five sites on the other side of each rabbit was injected. The appearance of each injection site was observed immediately after injection and at 24, 48 and 72 hours after injection. Individual animals were then observed daily for the signs of toxicity for 14 days. The tissue reaction for erythema, oedema was graded for each injection site and at each time interval. After 72 hours grading all erythema grades and oedema grades were totaled separately for each test substance and control. Each total was divided by 36 (6 animals×3 grading×2 grading categories) to determine the overall mean score for each test substance versus the control. The requirements of the test were met if the difference between the test and the control mean score is 1.0 or less. No adverse clinical signs or mortality was observed rabbits treated with the contact solution.

2. Tumorigenicity:

To ensure that the cells do not have any abnormalities/transformations that might lead to tumor formation in the recipient, tumorigencity assays were conducted in vitro. The ability of the cultured cells to form colonies in soft agar was assessed as an indicator of possible transformation and the potential ability for the cells to form tumors in humans. Cultures were monitored for 28 days for the formation of colonies of greater than 10 cells. No colonies were formed with the lots of cultured cells tested. FIG. 8 illustrates the melanocytes culture and FIG. 13 illustrates the results of keratinocytes co cultured along with melanocytes.

3. Transplantation in Animals:

Melanocytes (4×10⁴ cells/cm²) were seeded on PLA films and cultured for three days. At the time of transplantation, the cells were rinsed in HBSS and the films inverted on the wounds as mentioned below.

SCID mice (4 nos.) were anesthetized using a cocktail of 80 mg/kg ketamine, 40 mg/kg xylazine, and 0.05 mg/kg atropine i.p. Hair on the dorsal side was shaved and skin cleaned with 70% ethanol. Partial thickness wounds of 1 cm² were created on the dorsal side of animals under aseptic conditions. The wounds were rinsed with saline and the PLA films seeded with melanocytes were inverted on the wounds and covered with paraffin embedded gauze which was held in place with surgical plaster. Animals were housed individually and provided with food and water ad libitum. All animals were handled in accordance with the CPCSEA guidelines for the welfare of laboratory animals laid down by the Government of India. After 72 h, the animals were sacrificed and their wounds excised. The tissues were fixed in formalin and embedded in paraffin. Immunohistochemical staining to detect migrated melanocytes was performed on 5 μm thick sections using IHC HRP detection kit (Chemicon, USA). Briefly, sections were blocked with blocking buffer from the kit and then incubated with either monoclonal human nuclei antibody 1:50 (Chemicon, USA), or monoclonal MEL-5 antibody 1:50 (Signet, USA). Subsequently the samples were incubated with biotinylated goat anti-mouse secondary antibody followed by HRP Streptavidin present in the kit. Diaminobenzidine (Sigma, USA) was used as substrate to obtain a signal in positive cells. In addition, sections were stained with secondary antibody alone as control for non-specific binding of the secondary antibody.

4. Migration of the Cells from the Biopolymer

The ability of the keratinocytes and melanocytes to migrate into skin was tested in a full thickness punch wound biopsy model in guinea pigs and SCID mice respectively. Evidence for the migration of melanocytes from PLA films inverted on the wound bed was assessed in SCID mice as mentioned in point 4. The animals were sacrificed after 72 hours, the wounds were excised and processed for immunohistochemical examination.

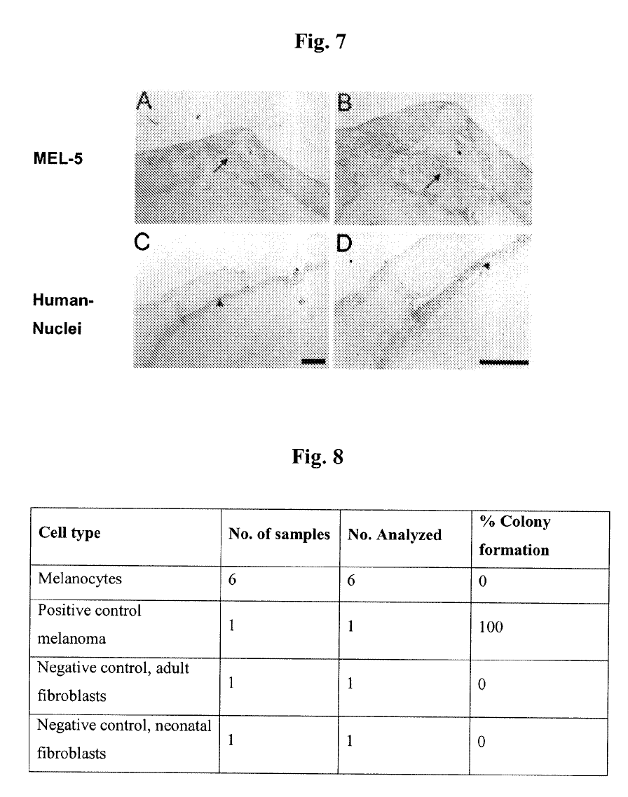

Histochemical analyses demonstrated that the cells had migrated into the wound bed and formed part of the regenerated epithelium. (FIG. 7). Melanocytes were identified by the presence of MEL-5 positive staining (FIGS. 7 A&B). MEL-5 positive cells were observed in the wound bed (arrow in FIGS. 7 A&B). Presence of migrated human melanocytes was verified by observing a similar staining pattern with anti-human nuclei antibody (FIGS. 7 C&D).

5. Further Studies

The following features are analyzed after transplantation.

1. Leaching

2. Color mismatch

3. Graft rejection

4. Scarring of the donor site

5. Post transplantation inflammation

6. Cobblestone appearance

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A graft composition capable of inducing pigmentation in skin comprising melanocyte cells derived from autologous epidermis and a biopolymer membrane, wherein at least 75% of the cells remain viable in the composition for at least 72 hours under transport conditions at 5-37° C.

2. The composition of claim 1, wherein, wherein at least 80% of the cells remain viable in the composition for at least 96 hours under transport conditions at 5-37° C.

3. The composition of claim 1, wherein the composition further comprises keratinocyte cells.

4. The composition of claim 3, wherein the composition the melanocyte and keratinocyte cells are present in a ratio between 0.8:2 to 1:1 on the biopolymer membrane.

5. The composition of claim 1, wherein the cells are present as a monolayer on the biopolymer membrane.

6. The composition of claim 5, wherein said monolayer is a sub-confluent monolayer.

7. The composition of claim 1, wherein the melanocytes cells are capable of further proliferation.

8. The composition of claim 1, wherein at least 90% of the cells are actively proliferating melanocytes.

9. The composition of claim 1, wherein the biopolymer membrane selected from the group consisting of polylactic acid (PLA), polyglycolic acid (PGA), and polylactic-polyglycolic acid copolymer (PLGA) having a molecular weight of at least 50,000 Da.

10. The composition of claim 1, wherein the biopolymer membrane is polylactic acid (PLA).

11. The composition of claim 1, wherein viable melanocyte cells are present in a sufficient quantity so that the resulting composition has a therapeutic effect against a hypo-pigmentation or hyper-pigmentation disorder of the skin.

12. A process for preparing the composition of claim 1 comprising the steps of:
a) isolating melanocyte cells from autologous epidermis;
b) preparing a cell suspension comprising the isolated melanocyte cells;
c) expanding the melanocyte cells;
d) optionally, cryopreserving the melanocyte cells;
e) optionally, thawing the melanocyte cells;
f) seeding the melanocyte cells onto a biopolymer membrane;
g) expanding the cells into a sub-confluent monolayer on the biopolymer membrane; and
h) transporting the biopolymer membrane using a transport device comprising transport media;
wherein at least 75% of the cells remain viable in the composition for at least 72 hours under transport conditions.

13. The process of claim 12, wherein the biopolymer membrane comprises polylactic acid (PLA), and the melanocyte cells are seeded onto the biopolymer membrane at a cell concentration of 1×10⁴ to 1×10⁵ cells/cm².

14. The process of claim 12, wherein the transport device comprises polycarbonate.

15. The process of claim 12, wherein the transport media comprises carbon dioxide enriched media.

16. The process of claim 12, wherein the transport media is KSFM media.

17. A method for treating an individual requiring skin pigmentation comprising applying the composition of claim 1 to the individual, wherein the composition enhances the rate of epidermal regeneration in the skin of the individual.

18. The method of claim 17, wherein the individual is human.

19. The method of claim 17, wherein the individual requiring skin pigmentation is suffering from vitiligo.

20. A composition comprising a sub-confluent monolayer of melanocyte cells and a biopolymer membrane, wherein at least 75% of the cells remain viable in the composition for at least 72 hours under transport conditions at 5-37° C.

21. A composition comprising a subconfluent monolayer of melanocytes co-cultured with keratinocytes and a biopolymer membrane, wherein at least 75% of the melanocyte cells remain viable in the composition for at least 72 hours under transport conditions at 5-37° C.

22. A composition of claim 21 wherein the melanocytes is cocultured in a ratio of 1:1 with keratinocytes.

23. A composition comprising (1) a sub-confluent monolayer of keratinocyte cells comprising undifferentiated cells and (2) a biopolymer membrane, wherein at least 75% of the cells remain viable in the composition for at least 72 hours under transport conditions at 5-37° C.

24. A method for treating hypopigmentation in a subject, comprising using a biopolymer membrane as a delivery system, and delivering to the skin a sub-confluent monolayer of melanocytes.

25. A method for treating hyperpigmentation in a subject, comprising using a biopolymer membrane as a delivery system, and delivering to the skin a sub-confluent monolayer of melanocytes

26. A kit for treating vitiligo, wherein the kit comprises (1) a composition comprising (a) a sub-confluent monolayer of melanocyte cells and (b) a biopolymer membrane, wherein the monolayer is on the biopolymer membrane; and (2) a transport device,
wherein at least 75% of the melanocyte cells remain viable in the composition for at least 72 hours under transport conditions at 5-37° C.
